(19) United States
(12) Patent Application Publication
(10) Pub. No.: US 2004/0241235 A1
(43) Pub. Date: Dec. 2, 2004

(54) Granules and granules coated with a masked taste

(76) Inventors: Christophe Lebon, rue de la Beneterie (FR); Sandrine Salle, Route de Rosny (FR); Pascal Supplie, rue du 8 Mai (FR)

Correspondence Address: Norman H Stepno, Burns Doane Swecker & Mathis, PO Box 1404, Alexandria, VA, 22313-1404, US

(21) Appl. No.: 10/471,234

(22) PCT Filed: Mar. 8, 2002
(86) PCT No.: PCT/FR02/00836

(30) Foreign Application Priority Data
Mar. 9, 2001 (FR) 01/03235

(51) Int. Cl. A61K 9/24

(52) U.S. Cl. 424/471

(57) ABSTRACT

Granules and coated granules, characterized in that they contain the following: a core containing at least one active ingredient which is optionally associated with at least one waxlike compound and optionally at least one polymer and/or binding agent; at least three successive layers of coating from the core outwards; a functional polymer coating (1) optionally containing a waxlike compound, enabling immediate, delayed or prolonged release, which can have a structure which is different from that of the first but which has a complimentary release function and which conditions the suspension medium.

The present invention relates to coated granules and granulates. It also relates to pharmaceutical presentations incorporating said coated granules or granulates.

The administration of solid oral forms such as tablets may prove to be dangerous, particularly for children and the elderly, who prefer chewable tablets, tablets that dissolve in the mouth or in a spoon of water, granules, powders, solutions or suspensions.

A number of active ingredients have an unpleasant taste, such that it is essential to mask their taste. Taste masking is defined as any method making it possible to delay or prevent the occurrence of an unpleasant taste specific to a product during its oral, buccal or nasal administration.

In the case of pharmaceutical formulations administered in dry forms, such as tablets, said masking must be maintained for at least the time that the product remains in the oral cavity, in order to improve comfort and optimise observance of the treatment by the patient.

In the case of formulations administered in liquid form, formulations packaged in multi-dose vial forms, particularly in the case of dry suspensions intended for extemporaneous reconstitution, also referred to as dry suspension for reconstitution, the lack of bitterness must be maintained for a time equivalent either to the treatment time or to the duration of the use of the vial. Therefore, the active granule or granulate used in such formulations should be stable in contact with an aqueous liquid phase for a period at least equal to 24 hours. In practice, this involves preventing the solubilisation of the active ingredient in the liquid phase.

In general, taste masking is carried out by encapsulating the active ingredient inside a capsule or by means of microencapsulation techniques wherein polymeric coating is applied to the active ingredient (WO 92/11871).

One of the solutions proposed consists of coating the active ingredient particles with a cellulose polymer. Said polymers particularly include ethylcellulose and hydroxypropylmethylcellulose.

Another solution consists of coating the active ingredient particle with an acrylic type polymer. Of said polymers, a distinction is made between pH-dependent polymers, i.e. polymers wherein the solubility depends on the pH and insoluble polymers wherein the intrinsic properties are not influenced by the pH of the medium.

However, even if the taste of the active ingredient present in the granules is masked in a satisfactory manner, said polymers interfere with the release of the active ingredient and require the use of agents favouring or delaying the solubilisation of the active ingredient (GB 1 511 85; WO 91/16043).

In addition, conventional techniques and formulas, although they provide satisfactory taste masking, do not make it possible to obtain stable membranes in suspension for more than one day.

Matrix microspheres have also been stabilised, but they require additional coating to achieve the desired stability; acceptable stability can be obtained in an acidic pH with cellulose acetates, but a delay in the release is observed (EP 0 293 885).

Therefore, there is still a great need to have a formulation enabling a rapid or controlled release of the active ingredient in a physiological medium, without said active ingredient being released in the formulation medium, which offers sufficient stability, i.e. an ability to retain the masking of the taste for a period at least equal to 24 hours.

The inventors surprisingly found that a granule or granulate comprising, firstly, a core containing an active ingredient possibly associated with at least one waxy compound and at least one polymer and, secondly, at least three coating layers, wherein the second contains at least one waxy compound, makes it possible to isolate the active ingredient for a sufficient time to ensure the stability of the masking of the taste when the dry suspension incorporating said coated granule or granulate is reconstituted by adding a defined volume of water when the first dose is administered. After administration, it is possible to have either an immediate release or a modified, i.e. delayed or sustained, release of the active ingredient.

Consequently, one of the aims of the present invention is to solve the problems, or at least improve the solutions implemented in the prior art to compensate for the difficulties in the development of this type of formulation.

Therefore, the present invention relates to coated granules and granulates characterised in that they comprise:

a core containing at least one active ingredient possibly associated with at least one waxy compound and possibly with at least one polymer and/or with at least one binding agent, and

at least three successive coating layers starting from the core:

a polymeric functional coating 1 possibly containing a waxy compound, enabling immediate, delayed or sustained release,

a hydrophobic coating 2 containing at least one waxy compound, and

a polymeric functional coating 3 possibly containing a waxy compound, which may have a different structure to the coating 1, but which has a complementary release function and conditions the suspension medium.

Within the scope of the present invention, the term immediate release refers to a release wherein the kinetics is not substantially modified by the formulation and/or by the parameters of the manufacturing method, which means that the dissolution profile of the active ingredient depends essentially on its intrinsic properties. On the other hand, the term modified release refers to a release wherein the kinetics is substantially modified by the formulation and/or by the parameters of the manufacturing method.

Within the scope of the present invention, the term complementary release function refers to a release of the same type as that obtained with the coating 1.

Within the scope of the present invention, conditioning the suspension medium signifies that the characteristics of the reconstituted suspension, obtained from excipient grains, are selected according to the release profile of the coated active granule or granulate, in vitro or after administration of said reconstituted suspension.

In a particular embodiment of the invention, additional layers may be applied wherein the composition is identical to that of layers 1 and 3.

An outer coating intended to mask any bitterness related to the constituents of the third coating layer 3, which does not modify the release properties of the coated granule and granulate substantially may be applied.

In a particularly advantageous embodiment of the invention, the core is a preferentially neutral substrate of determined grain size, based on starch, sucrose, ethylcellulose, lactose and wax, whereon the active ingredient is applied in a layer by atomising a suspension or a solution of said active ingredient, in an aqueous, organic solvent or in a mixture in the presence of at least one binding agent or at least one polymer or at least one waxy compound or a mixture of at least two of said agents and lubricants, if applicable.

In another advantageous embodiment of the invention, the core is the active ingredient itself, in the form of a spherical crystal or not, if its grain size allows effective direct coating. Otherwise, layer application (assembly) of the active ingredient should be carried out by atomising a solution or a suspension of said active ingredient in the presence of least one binding agent or at least one polymer or at least one waxy compound or a mixture of at least two of said agents and lubricants, if applicable, and organic solvents or water.

In another particularly advantageous embodiment of the invention, the core is a granulate based on the active ingredient obtained by granulation. The granulate may be obtained by wet granulation or in a fluidised air bed, or by spherical crystallisation or by emulsion-diffusion of solvent preferentially using (a) solutions of granulations based on organic solutions of waxy compound(s) in the presence of lubricants and plasticisers or (b) a polymer such as hydroxypropylmethylcellulose. In addition, assembly of the active ingredient may be carried out using said granulate as a substrate, by atomising a solution or suspension of active ingredient in organic solvents or in water, in the presence of at least one binding agent or at least one polymer or at least one waxy compound or a mixture of at least two of said agents and lubricants, if applicable.

In addition to the active ingredient, the core may contain various agents; these agents include insoluble agents, particularly talc, silicone dioxide, titanium dioxide, silica, alumina, starch and mixtures thereof; they also include soluble agents, particularly mannitol, sucrose, lactose, dextrose, sodium chloride, sorbitol and mixtures thereof, polyethylene glycol or amphiphilic compounds (magnesium stearate, polysorbates).

The core may contain up to 100% active ingredient, preferentially between 30 and 85% according to the dosage of the final formulation and the proportion of dry content to obtain a homogeneous suspension.

The core containing the active ingredient may have any suitable size, but preferentially the size distribution of the core containing the active ingredient has a mean between 100 and 500 μm, the mean being preferentially between 100 and 250 μm when the core is a granulate or the active ingredient itself, and preferentially between 400 and 500 μm when the core is a neutral substrate whereon the active ingredient is applied in a layer.

As active ingredients, it is particularly possible to use, without this list being restrictive: antacids, anti-inflammatories, coronary or peripheral vasodilators, anti-infectious agents, antibiotics, antiparasitics, anxiolytics, psychotropic agents, neuroleptics, central nervous system stimulants, antihistamines, antidiarrhoeals, nutritional supplements, antivirals, antispasmodics, vasoconstrictors, antithrombotics, antimigraine agents, analgesics, antipyretics, antiasthmatics, antitussives, mucoregulators, decongestants, plant extracts and antinauseants.

Preferentially, the active ingredient is an anti-infectious substance, selected from the macrolides.

The latter particularly include erythromycin and its derivatives, and clarithromycin.

According to the invention, the coatings 1 and 3 are functional coatings, wherein the purpose is to provide an active ingredient release property, that is immediate, sustained or delayed; they consist of polymers conventionally known to those skilled in the art to provide said properties possibly associated with a waxy compound. Delayed-release polymers particularly include: polymethacrylates particularly those marketed under the name Eudragit®L, Eudragit®S and Eudragit® FS30D, cellulose acetophthalate and cellulose acetate; sustained-release polymers include: polymethacrylates particularly those marketed under the name Eudragit® NE, Eudragit®RS and Eudragit®RL, ethyl cellulose, polyvinyl acetate, polyvinyl alcohol and copolymers thereof; immediate-release polymers include: polymethacrylates particularly those marketed under the name Eudragit®E.

The waxy compounds used may particularly be selected from the group consisting of: waxes, Novata® waxes, gelucires and suppocires, glycerol macrogols, fatty acids (stearic acid), fatty acid esters, glycerol monostearate Precirol®, Compritol®.

Of these waxy compounds, hydrophobic waxy compounds are advantageously used and hydrophobic waxy compounds with a low HLB (hydrophilic-lipophilic balance) and with a melting point between 35 and 53° C., preferentially between 37 and 43° C., even more advantageously. These include, in a non-restrictive manner, the waxy compounds marketed under the names Gelucire® 43/01 and Novata®AB.

These waxy compounds may be associated with glycerol monostearate (GMS).

In this way, if an immediate release is desired, it is possible to use as functional coatings 1 and 3, a coating consisting advantageously of a mixture of Eudragit® E100 and possibly hydrophobic waxy compounds with a low HLB (hydrophilic-lipophilic balance) and with a melting point between 35 and 53° C., preferentially between 37 and 43° C. in the presence of lubricants. These include, in a non-restrictive manner, Gelucire® 43/01 and Novata®AB, possibly associated with glycerol monostearate (GMS).

If a delayed release is desired, it is possible to use as functional coatings 1 and 3, a coating based on an aqueous dispersion or an organic solution of Eudragit® L in the presence of hydrophobic plasticisers and lubricants.

If a modified release is desired, the functional coatings 1 and 3 may be based on an aqueous dispersion or an organic solution of ethylcellulose or Eudragit® RL or RS or a coating based on an organic solution of said polymers or Eudragit® S in the presence or not of waxy compounds and/or lubrication agents, plasticisers and lubricants.

The coating contents for the coating 1 (calculated as a percentage (w/w) of dry content applied to the initial substrate) is advantageously between 5 and 100% and preferentially between 30 and 60%.

The purpose of the hydrophobic coating 2 is to increase the stability of the suspended grain. It consists of a waxy compound solution base in a solvent and possibly comprises a lubrication agent such as, for example, talc, hydrophobic colloidal silica or glycerol monostearate (GMS). The coating content for said second coating (calculated as a percentage (w/w) of dry content applied to the initial substrate) is advantageously between 5 and 100% and preferentially between 20 and 80%.

In this way, this hydrophobic coating 2 advantageously comprises a waxy compound or a combination of low-HLB hydrophobic waxy compounds and with a melting point between 35 and 53° C., preferentially 37 and 43° C. in a solvent. This particularly includes Gelucire® 43/01, Gelucire® 53/01, Novata® AB, glycerol monostearate and mixtures thereof.

The polymeric function coating 3 which has complementary release functions to those of the coating 1 is either identical or analogous to said coating 1, but has the same properties with respect to the release of the active ingredient and conditions the suspension medium. The coating content on said coating 3 (calculated as a percentage (w/w) of dry content applied to the initial substrate) is advantageously between 5 and 200% and preferentially between 80 and 160%.

If the coating 3 has a strong taste due to the excipients comprised therein, then an outer coating based on Eudragit® RL30D and RS30D or mixtures thereof, in the presence of plasticisers and lubricants, is applied. The coating content at this level will advantageously be between 0 and 15% and preferentially between 0 and 5%.

The lubrication agents (lubricant agents) are advantageously selected from the group comprising talc, hydrophobic colloidal silica and glycerol monostearate.

The plasticisers are advantageously selected from the group consisting of dibutylsebaccate, triethylcitrate, diethylphthalate, acetyltriethylcitrate, acetyltributylcitrate, glycerol monostearate (GMS) and Myvacet®.

The coated granules and granulates according to the invention are prepared according to a method which comprises the production of the core or substrate and possibly includes an additional assembly step.

The method may advantageously comprise the following steps:

application of the solubilised active ingredient onto the substrate, in the presence of preferentially hydrophobic waxy compounds and/or polymers, and at least one lubrication agent in a solvent or a solvent mixture,

application of a first coating, polymeric functional coating 1 and possibly waxy compounds, said coating enabling an immediate, delayed or sustained release,

application of a second coating, hydrophobic coating 2 containing at least one waxy compound or a combination of waxy compounds,

application of a third coating, polymeric functional coating 3 and possibly waxy compounds, said coating liable to have a different structure to that of the coating 1, but having a complementary release function, and if applicable

drying of the granules or granulates obtained in this way.

The coating solvents are those conventionally used by those skilled in the art. For example, these include water, methylene chloride, ethanol, isopropanol and mixtures thereof.

Said method is carried out in a fluidised air bed or by means of any other similar industrial method known to those skilled in the art.

The drying operation may be carried out in a fluidised air bed, in a vacuum rotary drier or by means of any equivalent technique enabling the removal of the residual solvents.

According to an advantageous embodiment of the invention, the method also comprises the application of additional layers identical to the layers 1 and 3 and essentially the application of an outer coating aiming to mask the taste of the constituents of the preceding coating.

The coated granules and granulates according to the invention may be used in any suitable pharmaceutical formulation enabling immediate reconstitution in a liquid medium. They may particularly be used to prepare dry syrups, tablets, sachets and suspensions. Of said suspensions, dry suspensions for reconstitution, i.e. powders packaged in multi-dose vials which can be reconstituted before use as a suspension in a liquid such as water, should advantageously be selected.

The powders for reconstitution prepared from granules and granulates according to the invention are stable during storage and the suspensions, once reconstituted in the multi-dose vial, have a masked taste for the duration of the treatment or, if the treatment requires several vials, for the duration of the use of the vial. In any case, the reconstituted suspension is stable for at least 24 hours. These suspensions also have a sufficient bioavailability and are particularly useful in paediatric and geriatric treatments.

The invention also relates to a dry suspension for reconstitution containing granules or granulates according to the invention.

In said formulation, the active grain gives the suspension its taste masking and release properties.

This dry suspension for reconstitution also contains excipients giving the reconstituted formulation particular organoleptic characteristics and also microbiological stability.

These excipients are selected from those conventionally used by those skilled in the art to produce said formulations. These excipients include sweeteners, colorants, viscosity agents or thickeners, pH-modulating agents, preservatives (antimicrobial or fungicidal), surfactants and antioxidants.

This suspension may be obtained in several ways:

by simply adding excipients in powder form to the active grain,

by adding a dry excipient granulate to the active grain. In this case, the excipients are granulates preferentially obtained by wet granulation,

by adding to the active grain excipients assembled on the active grain by means of a coating method carried out advantageously in a fluidised air bed.

In addition, the invention also relates to a dry mixture comprising granules or granulates according to the present invention associated with any suitable excipient to obtain a dry suspension for reconstitution in a liquid medium wherein at least one is a thickening agent, one is a preservative and one is a pH-modulating agent.

Examples of thickeners include all the thickeners known to those skilled in the art, particularly those selected from the group comprising gums such as xanthan, guar and traganth, magnesium silicate and combinations thereof, sodium alginate, propylene glycol alginate, cellulose compounds such as hydroxyethyl cellulose, hydroxypropyl cellulose, methyl cellulose, carboxymethyl cellulose, carbomers, gelatine, poloxamers, or combinations of these compounds and carrageenans.

Examples of pH-adjusting agents advantageously include those selected from the group comprising citric acid, soda, sodium citrate, trisodium citrate or any other pharmaceutically acceptable compound having the ability to buffer an aqueous solution.

Examples of preservatives include those selected from the group comprising potassium or sodium sorbate, sodium benzoate, azorubin, bronopol, ethylene diamine tetra-acetic acid (EDTA), methyl, ethyl, propyl and butyl p-hydroxybenzoate (parabens) and salts thereof, used alone or in a mixture, propionic acid, sulphites and cresol.

The suspension may also contain one or more sweeteners such as saccharin salts and/or potassium acesulfam, or any other sweetener known to those skilled in the art such as aspartame, sucrose and its derivatives, trehalose, sodium glycyrrhizinate or mixtures thereof, an opacifying agent such as Opadry® OYB or titanium oxides and product capture agents such as cyclodextrins wherein the quantities are adapted according to the size of the molecule and the function to be isolated.

The suspension may also contain one or more aromatic compositions and a filling agent, particularly polyols, for example sorbitol (Neosorb®), xylitol and lactitol.

The excipient grain may be obtained by means of a wet granulation method or any other similar industrial method known to those skilled in the art. It may also be obtained by producing a hydro-alkanol solution of the sweeteners and/or preservatives to be used as a wetting solution with a mixture of filling agents such as sorbitol, thickening agent, opacifying agent, pH-adjusting agent, aromatic formulations if applicable, the purpose of the filling agent being to create a sufficient mass for the granulation. Any other excipient fulfilling the same function may also be used.

Another alternative consists of assembling the excipients on the active grain by any technique known to those skilled in the art, particularly in a fluidised air bed.

When the first dose of medicinal product is administered, the suspension is prepared by adding a defined quantity of water (for example, by volume, or using a mark on the vial), directly into the vial containing the final dry mixture.

The excipient grains prepared in this way enable rapid reconstitution of the suspension, which only requires manual stirring by turning to homogenise the preparation; in addition, the suspension obtained has a good bacteriological stability and masking stability of over 7 days, independent of the pH of the suspension. It is particularly useful in paediatric and geriatric treatments.

The pH of the suspension is adjusted according to the properties of the coated granule or granulate to be associated.

If an immediate release is desired, the pH of the suspension should be between 5.5 and 10, preferentially between 8.5 and 10. For a delayed release, the pH of the suspension should be between 3 and 7, preferentially between 4 and 5.

Due to the presence of waxy agents, the masking stability of the suspensions is enhanced. The waxy agents also make it possible to reduce the quantity of polymers used for the coating and therefore the toxicity induced by said polymers.

The invention and the advantages it offers will be seen more clearly using the examples of embodiments given below.

### EXAMPLE 1

### Immediate-Release Clarithromycin Suspension (CHL 13.05)

1.1 Active Grain Preparation: since the CHL 13.05 used has a fine grain size distribution, granulation followed by assembly will be carried out.

Step 0: a mixture of powders was produced and placed in the fluidised air bed vessel:

| | |
|---|---|
| CHL 13.05: | 71.4% |
| Aerosil ® R972: | 7.1% |
| Talc M 10: | 21.5% |

Step 1a: granulation

A solution based on Gelucire® 43/01—Aerosil® R 972 (81%-19%) in methylene chloride is atomised onto the powder mixture.

The dry concentration in methylene chloride is equal to 10% by weight and the dry atomised/substrate ratio is equal to 37.5% by weight.

Step 1b: assembly

A solution based on CHL 13.05—Gelucire® 43/01—Talc M 10 (51.7%-34.5%-13.8%) in a methylene chloride-ethanol mixture (77.9%-22.1% by weight) is atomised onto the granulate obtained in step 1a.

The dry concentration in methylene chloride-ethanol is equal to 11.9% by weight and the dry atomised/substrate ratio is equal to 100% by weight.

Step 2: coating 1=polymeric functional coating

A solution based on Eudragit® E 100—Gelucire® 43/01—Talc M (10/1) (51.4%-5.7%-42.9%) is atomised onto the granulate obtained in step 1b.

The dry concentration in methylene chloride is equal to 12.9% by weight and the dry atomised/substrate ratio is equal to 52.5% by weight.

Step 3: Coating 2=hydrophobic coating

A solution based on Gelucire® 43/01—Talc M 10 (57.1%-42.9%) in methylene chloride is atomised onto the granulate obtained in step 2.

The dry concentration in methylene chloride is equal to 18.2% by weight and the dry atomised/substrate ratio is equal to 35% by weight.

Step 4: coating 3=polymeric functional coating

A solution based on Eudragit® E 100—Gelucire® 43/01—Talc M (10/1) in a methylene chloride water (10/1) mixture is atomised onto the granulate obtained in step 3.

The dry concentration in methylene chloride is equal to 12.9% by weight and the dry atomised/substrate ratio is equal to 105% by weight.

1.2. Preparation of Grain for Suspension

| Excipient | Quantity (g) |
|---|---|
| Sorbitol | 400 |
| (Neosorb ® P100T) | |
| Carrageenan | 48.7 |
| (Viscarin ® GP 209) | |
| Aromatic formulation | 24.9 |
| Citric acid | 0.7 |
| Opadry ® OYB | 48.7 |

Wetting solution

| | |
|---|---|
| Sodium saccharinate | 4.2 |
| Total parabens | 14 |
| (Sodium Nipasept ®) | |
| Potassium acesulfam | 1.0 |
| Purified water | 35.1 |
| Ethanol 96 BG | 35.1 |

1.3. Distribution and Reconstitution of Suspension

30% of excipient grains and 70% of active grains are introduced into the final packaging (by mixing followed by single feeding or double feeding with no prior mixing). The vial is filled according to the dose of CHL 13.05 administered for the treatment. At the moment of use, fill up to the mark with mineral water. The reconstituted suspension is stable for at least 7 days.

### EXAMPLE 2

### Delayed-Release Clarithromycin Suspension (CHL 13.05) (Enteric Suspension)

2.1. Active Grain Preparation:

Step 1: the grain constitution steps are similar to those in the previous example.

Step 2: coating 1=polymeric functional coating

A solution based on Eudragit® L30D (dry extract)—Myvacet 9.45—Talc M 10 (77%-11.5%-11.5%) diluted in purified water is atomised onto the granulate obtained in step 1.

The dry concentration in total water is equal to 32.6% by weight and the dry atomised/substrate ratio is equal to 39% by weight.

Step 3: Coating 2=hydrophobic coating

A solution based on Gelucire® 43/01—Talc M 10 (57.1%-42.9%) in methylene chloride is atomised onto the granulate obtained in step 2.

The dry concentration in methylene chloride is equal to 19.4% by weight and the dry atomised/substrate ratio is equal to 35% by weight.

Step 4: coating 3=polymeric functional coating

A solution based on Eudragit® L30D (dry extract)—Myvacet 9.45—Talc M 10 (71.4%-10.7%-17.9%) diluted in purified water is atomised onto the granulate obtained in step 3.

The dry concentration in total water is equal to 34.5% by weight and the dry atomised/substrate ratio is equal to 154% by weight.

Step 5: outer coating

A solution based on Eudragit® S100—Myvacet 9.45—Talc M 10 (83.3%-8.3%-8.3%) in ethanol is atomised onto the granulate obtained in step 4.

The dry concentration in ethanol is equal to 9.8% by weight and the dry atomised/substrate ratio is equal to 0.6% by weight.

2.2. Preparation of Grain for Suspension

| Excipient | Quantity (g) |
|---|---|
| Sorbitol | 400 |
| Neosorb ® P100T | |
| Carrageenan | 49.1 |
| Viscarin ® GP 209 | |
| Aromatic formulation | 25.1 |
| Citric acid | 14.5 |
| Opadry ® OYB | 49.1 |

Wetting solution

| | |
|---|---|
| Sodium saccharinate | 4.3 |
| Total parabens | 10.5 |
| (Sodium Nipasept ®) | |
| Potassium acesulfam | 1.1 |
| Purified water | 35.1 |
| Ethanol 96 BG | 35.1 |

2.3. Distribution and Reconstitution of Suspension

The mixture consists of 75% excipient grains and 25% active grains and is then treated as for the previous example.

### EXAMPLE 3

### Solubility and Stability Tests

The stability of the granulates prepared according to the procedure in examples 1 and 2 is evaluated in terms of degradation products, dissolution kinetics, taste and residual solvents.

The stability of the suspensions obtained from granulates prepared according to the procedure in examples 1 and 2, is evaluated in terms of pH, taste masking and released active ingredient assay.

The results are contained in the table below:

| | |
|---|---|
| Example 2 | Example 1 |

Granulation

| | |
|---|---|
| D₁₀ (μm) | 25 |
| D₅₀ (μm) | 80 |
| D₉₀ (μm) | 185 |

Assembly

| | |
|---|---|
| D₁₀ (μm) | 70 |
| D₅₀ (μm) | 160 |
| D₉₀ (μm) | 290 |

Polymeric functional coating 1

| | | |
|---|---|---|
| D₁₀ (μm) | 100 | 110 |
| D₅₀ (μm) | 195 | 240 |
| D₉₀ (μm) | 330 | 400 |

Hydrophobic coating 2

| | | |
|---|---|---|
| D₁₀ (μm) | 140 | 160 |
| D₅₀ (μm) | 240 | 320 |
| D₉₀ (μm) | 380 | 600 |

Polymeric functional coating 3

| | | |
|---|---|---|
| D₁₀ (μm) | 220 | 260 |
| D₅₀ (μm) | 330 | 470 |
| D₉₀ (μm) | 550 | 710 |
| Dissolution | | |
| HCl (2 h) | 0% | NP |
| pH 6.8 (1 h) | 43.9% | 93.9% |
| pH 6.8 (2 h) | 63.2% | NP |
| Residual solvents | | |
| before drying: | | |
| Ethanol | 347 ppm | 355 ppm |
| CH₂C₁₂ | 9 ppm | 1065 ppm |
| Water | 1.5% | NP |

Stability studies
Dry suspension for reconstitution

| | | |
|---|---|---|
| Stability | At least 2 months | At least 2 months |
| 25° C./60% RH | | |
| Stability | At least 2 months | At least 2 months |
| 25° C./60% RH | | |

Reconstituted suspension

| | | |
|---|---|---|
| Reconstitution at | Stable for 14 | Stable for 14 |
| ambient | days | days |
| temperature | | |

The above table shows that:

both formulations are stable over time,

the masking of the taste is sustained, and

the dissolution profiles are satisfactory.

1. Coated granules and granulates comprising:
a core comprising at least one active ingredient selected from anti-infectious substances, optionally associated with at least one waxy compound and with at least one polymer and/or with at least one binding agent, and
at least three successive coating layers starting from the core:
a polymeric functional coating (1) optionally comprising a waxy compound, enabling immediate, delayed or sustained release,
a hydrophobic coating (2) comprising at least one waxy compound, and
a polymeric functional coating (3) optionally comprising a waxy compound, which may have a different structure to the coating (1), but which has a complementary release function and conditions the suspension medium.

2. Coated granules and granulates according to claim 1, wherein coating contents are respectively between 5 and 100% for the coating (1), between 5 and 100% for the coating (2) and between 5 and 200%—for the coating (3).

3. Coated granules and granulates according to claim 1, wherein the core is a neutral substrate whereon the active ingredient is applied in a layer.

4. Coated granules and granulates according to claim 1, wherein the core is the active ingredient itself, in the form of a spherical crystal or not.

5. Coated granules and granulates according to claim 1, wherein the core is a granulate based on the active ingredient obtained by granulation.

6. Coated granules and granulates according to claim 1, wherein, in addition to the active ingredient, the core comprises various agents.

7. Coated granules and granulates according to claim 1, wherein the core comprises up to 100% active ingredient.

8. Coated granules and granulates according to claim 1, wherein the size distribution of the core has a mean between 100 and 500 μm.

9. Coated granules and granulates according to claim 3, wherein the size distribution of the core has a mean between 400 and 500 μm.

10. Coated granules and granulates according to claim 4, wherein size distribution of the core has a mean between 100 and 250 μm.

11. Coated granules and granulates according to claim 1, wherein the anti-infectious substance comprises one or more of the macrolides.

12. Coated granules and granulates according to claim 11, wherein the macrolide comprises erythromycin, derivatives thereof, or clarithromycin.

13. Coated granules and granulates according to claim 1, wherein the polymers providing delayed-release properties comprise polymethacrylates, cellulose acetophthalate or cellulose acetate.

14. Coated granules and granulates according to claim 13, wherein the polymethacrylate is selected from the group consisting of Eudragit®L, Eudragit®S, Eudragit® FS30D and mixtures thereof.

15. Coated granules and granulates according to claim 1, wherein the polymers providing sustained-release properties comprise polymethacrylates, ethyl cellulose, polyvinyl acetate, polyvinyl alcohol or copolymers thereof.

16. Coated granules and granulates according to claim 15, wherein the polymethacrylate is selected from the group consisting of Eudragit® NE, Eudragit® RS, Eudragit® RL and mixtures thereof.

17. Coated granules and granulates according to claim 1, wherein the polymer providing immediate-release properties is a polymethacrylate, advantageously Eudragit® E.

18. Coated granules and granulates according to claim 1, wherein the waxy compounds are selected from the group: consisting of waxes, Novata® waxes, gelucires and suppocires, glycerol macrogols, fatty acids (stearic acid), fatty acid esters, glycerol monostearate, Precirol® and Compritol®.

19. Coated granules and granulates according to claim 18, wherein the waxy compounds are hydrophobic waxy agents with a low HLB (hydrophilic-lipophilic balance) and with a melting point between 35 and 53° C.

20. Coated granules and granulates according to claim 19, wherein the waxy compounds are associated with glycerol monostearate.

21. Coated granules and granulates according to claim 20, wherein the waxy compounds are Gelucire® 43/01 and/or Novata®AB, optionally associated with glycerol monostearate.

22. Coated immediate-release granules and granulates according to claim 1, wherein the functional coatings (1) and (3) comprise a mixture of Eudragit® E100 and hydrophobic waxy compounds with a low HLB (hydrophilic-lipophilic balance) and with a melting point between 35 and 53° C.—in the presence of lubricants.

23. Coated granules and granulates according to claim 22, wherein the waxy agents are Gelucire® 43/01 and/or Novata®AB, optionally associated with glycerol monostearate.

24. Coated delayed-release granules and granulates according to claim 1, wherein the functional coatings (1) and (3) are based on an aqueous dispersion or an organic solution of Eudragit® L in the presence of hydrophobic plasticisers and lubricants.

25. Coated modified-release granules and granulates according to claim 1, wherein the functional coatings (1) and (3) are based on an aqueous dispersion or an organic solution of ethylcellulose or Eudragit® RL or RS or a coating based on an organic solution of said polymers or Eudragit® S in the presence or not of waxy compounds and/or lubrication agents, plasticisers and lubricants.

26. Coated granules and granulates according to claim 1, wherein the coating solvents are selected from the group consisting of water, methylene chloride, ethanol, isopropanol and mixtures thereof.

27. Coated granules and granulates according to claim 1, wherein the lubrication agents are selected from the group consisting of talc, hydrophobic colloidal silica and glycerol monostearate.

28. Coated granules and granulates according to claim 1, wherein the plasticisers are selected from the group consisting of dibutylsebaccate, triethylcitrate, diethylphthalate, acetyltriethylcitrate, acetyltributylcitrate, glycerol monostearate and Myvacet®.

29. A method for enabling immediate reconstitution of solids in a pharmaceutical formulation comprising using the coated granules and granulates according to claim 1, in any suitable pharmaceutical formulation.

30. The method according to claim 29, wherein the pharmaceutical formulation is a dry suspension for reconstitution.

31. Dry mixture comprising granules and granulates according to claim 1, associated with any suitable excipient to obtain a dry suspension for reconstitution in a liquid medium wherein at least one is a thickening agent, one is a preservative and one is a pH-modulating agent.

32. Dry suspension for reconstitution comprising granules and granulates according to claim 1.

33. Suspension obtained by adding a defined quantity of water using a dry suspension for reconstitution according to claim 32.

34. Immediate-release suspension according to claim 33, wherein the pH is between 5.5 and 10.

35. Delayed-release suspension according to claim 33, wherein the pH is between 3 and 7.

36. Method to prepare coated granules and granulates according to claim 1, comprising the production of the core or substrate and includes an optional additional assembly step.

37. Method according to claim 36, comprising the following steps:
application of the solubilised active ingredient onto the substrate, in the presence of preferentially hydrophobic waxy compounds and/or polymers, and at least one lubrication agent in a solvent or a solvent mixture,
application of a first coating, polymeric functional coating (1) and optionally waxy compounds, said coating enabling an immediate, delayed or sustained release,
application of a second coating, hydrophobic coating (2) comprising at least one waxy compound or a combination of waxy compounds,
application of a third coating, polymeric functional coating (3) and optionally waxy compounds, said coating liable to have a different structure to that of the coating (1), but having a similar release function, and optionally,
drying of the granulates.

38. The coated granules and granulates according to claim 2, wherein coating contents are respectively between 30 and 60% for the coating (1), between 20 and 80% for the coating (2) and between 8 and 160% for the coating (3).

39. The coated granules and granulates according to claim 7, wherein the core comprises 30 to 85% active ingredient.

40. The coated granules and granulates according to claim 19, wherein the waxy compounds have a melting point between 37° and 43° C.

41. The coated immediate-release granules and granulates according to claim 22, wherein the functional coatings (1) and (3) comprise a mixture of Eudragit® E100 and hydrophic waxy compounds with a melting point between 37° and 43° C. in the presence of lubricants
